(19) United States
(12) Patent Application Publication
(10) Pub. No.: US 2022/0169596 A1
(43) Pub. Date: Jun. 2, 2022

(54) DERIVATIVES OF 10-METHYLENE LIPIDS, PROCESS FOR PREPARING SUCH DERIVATIVES AND USE THEREOF

(71) Applicants: Jean-Christophe RABOIN, CHAUMONT SUR THARONNE (FR); Henri STRUB, Pont Sainte Maxence (FR); GINKGO BIOWORKS, INC., Boston, MA (US)

(72) Inventors: Jean-Christophe RABOIN, Chaumont Sur Tharonne (FR); Henri STRUB, Pont Sainte Maxencefr (FR)

(21) Appl. No.: 17/593,759

(22) PCT Filed: Mar. 26, 2020
(86) PCT No.: PCT/EP2020/058484

§ 371 Date: Sep. 24, 2021

(30) Foreign Application Priority Data
Mar. 26, 2019 (EP) 19305385.7

(51) Int. Cl. C07C 229/08; C07C 53/19; C07C 59/01; C07C 227/08; C07C 51/367; C07C 51/363; C08G 69/06; C07D 313/00; C07D 225/02; A01N 37/36

(52) U.S. Cl. CPC C07C 229/08; C07C 53/19; C07C 59/01; C07C 227/08; C07C 51/367; A01P 3/00; C08G 69/06; C07D 313/00; C07D 225/02; A01N 37/36; C07C 51/363

(57) ABSTRACT

The present invention relates to derivatives of 10-methylene lipids, their preparation and their use.

### FIELD OF THE INVENTION

The present invention relates to derivatives of 10-methylene lipids, their preparation and their use.

### BACKGROUND OF THE INVENTION

Tuberculostearic acid (10-methylstearic acid) and corresponding 10-methylene biological precursor are known, see, e.g. Yuzuru Akamatsu and John Law, “Enzymatic synthesis of 10-methylene stearic acid and tuberculostearic acid”, Biochemical and Biophysiscal Research Communications, Vol. 33, Issue 1, 10^{th} Oct. 1968, pages 172-176.

Even if authors state that tuberculostearic acid is a main component of the phospholipids from Mycobacterium species, its low production yield related to total lipid content prevented further industrial use. Recently, Novogy, Inc. was able to insert and to express gene sequences responsible for methylation of fatty acids into Yarrowia lipolytica (Shaw et al., “Heterologous production of 10-methyl stearic acid”, WO2018/057607).

Although the production of 10-methylene biological precursor of tuberculostearic acid can now be envisaged at larger scale from genetically modified cells, stability of such precursor does not allow a broad range of purification methods which would be suitable for industrial scale production at reasonable cost. There is a need to provide stable and easy to use starting material based on 10-methylene stearic acid for a wide range of applications.

### SUMMARY OF THE INVENTION

The present invention provides tuberculostearic acid derivatives which are more versatile synthetic intermediates for a wide range of applications, in particular for the preparation of polyamides, polyesters, block copolymers such as PEBA (Polyether Block Amide), lubricants such as for Jet engines with improved properties when tested against SAE AS5780 High Performance Capability (HPC) standard, starting material for flavors and fragrances industry, pharmaceutical drugs and agrochemicals. Preferred applications are polymers, lubricants and as oil or gas well drilling and completion aid. Alternatively, preferred applications are for cosmetics and for flavors and fragrances.

According to a first aspect, the invention provides an intermediate product for preparing a product chosen at least from polyamides, polyesters, lactams and lactones, preferably at least from polyamides, polyesters, lactams and lactones, flavors, fragrances, pharmaceutical drugs, agrochemicals.

The intermediate product of the present invention has a saturated aliphatic linear chain of 14-20 carbon atoms, said aliphatic linear chain having
a terminal group chosen from a carboxylic acid and a carboxylic acid ester,
a methyl group branched in the Δ10 position,
a functional group bound to the carbon of the said branched methyl group, this functional group being chosen from bromo (—Br), hydroxyl (—OH) and amino (—NH₂) groups.

Particularly preferred functional group bound to the carbon of the said branched methyl group is bromo, since bromo may be easily converted into hydroxyl or amino groups.

Said intermediate product is remarkable in that it is easy to use due to its stability and allows the preparation of many further products, including, but not limited to, the above mentioned polyamides, polyesters, lactams and lactones, and also as starting material for flavors and fragrances industry, pharmaceutical drugs and agrochemicals.

Miller et al. in JAOCS Vol. 51, No. 10, pages 427-432, 1 Oct. 1974, describe the preparation of a mixture of 9-aminomethyloctadecanoic and 10-aminomethyloctadecanoic acids in two steps starting from oleonitrile. This method comprises an hydroformylation step involving the use of costly rhodium based catalysts, followed by oxidation and amination. Hydroformylation is not regioselective so that 9- and 10-aminomethyl isomers are simultaneously produced. They are not separated and used as such for (co-)polymerization to make polyamides. No method for monomers purification is provided.

Similarly, Behr et al. in Eur. J. Lipid Sci. Technol. 102 (2000), 467-471 prepare the same kind of derivatives with the same hydroformylation method, resulting in the same drawbacks as per isolation of pure products. Products are always analyzed under the form of mixtures. Methylamino groups are always substituted.

WO2013/142206 discloses Guerbet alcohols and method for preparing the same, wherein those Guerbet alcohols are always bearing two terminal carboxylic acids or esters, when present.

WO2017/001194 discloses a one-pot synthesis of hydroxymethylated trigycerides from triglycerides which comprise at least one carbon-carbon double bond along one of the fatty acid ester chains thereof. Hydroxymethylation of double bonds is performed using rhodium or cobalt catalyst under CO and H₂ pressure.

Optionally, the aliphatic linear chain has 16, 17 or 18 carbon atoms, preferably 18 carbon atoms.

Optionally, the terminal group is an ester chosen from methyl, ethyl, propyl and isopropyl esters.

According to a second aspect, the invention provides a composition containing at least one intermediate product of the present invention.

Optionally, said composition may contain other products, for example other lipids, in particular originated from cell fermentation, optionally having a functional group chosen from the same list defined above for the intermediate product of the invention.

According to a further aspect, the invention provides a process for preparing the intermediate product or the composition of said invention, said process comprising the following steps:
a) providing at least one 10-methylene-substituted lipid comprising an aliphatic linear chain of 14-20 carbon atoms having a methylene (═CH₂) group branched in the Δ10 position and a terminal group chosen from a carboxylic acid and a carboxylic acid ester, optionally in mixture with other lipids,
b) reacting said at least one 10-methylene-substituted lipid, optionally in mixture with other lipids, with at least one reagent under conditions to provide at least one corresponding 10-methyl-substituted lipid, optionally in mixture with said other lipids, said corresponding 10-methyl-substituted lipid comprising:
a saturated aliphatic linear chain with the same number of carbon atoms than the aliphatic linear chain of the methylene-substituted lipid,
a methyl group branched in the Δ10 position, and
a functional group bound to the carbon of the branched methyl group, chosen from bromo (—Br), hydroxyl (—OH) and amino (—NH₂) groups.

Such process allows the recovery of a stable 10-methyl-substituted lipid which can be further used in the preparation of above mentioned products.

In one embodiment, step a) may provide a single 10-methylene-substituted lipid, i.e. a pure product. In such a case, a single corresponding 10-methyl-substituted lipid is recovered from step b), optionally with by-products.

In another embodiment, step a) may provide two or more 10-methylene-substituted lipids. In such a case, two or more corresponding 10-methyl-substituted lipids are recovered from step b).

In still another embodiment, step a) may provide one or several 10-methylene-substituted lipids in mixture with other lipids. In this case, the 10-methyl-substituted lipids issued from the corresponding methylene-substituted lipids are recovered from step b) in mixture with said other lipids, which may also have reacted with the reactant if they have one or more carbon-carbon double bonds.

In the last two embodiments, a composition according to the invention is recovered from step b).

Advantageously, the 10-methylene-substituted lipid(s) provided in step a) may be obtained by microbial production of bio-organic compounds. In particular, such 10-methylene-substituted lipid(s) can be fermentation products produced by microorganisms, for example genetically modified microorganisms. Methylene-substituted lipid(s) in mixture with other lipids may in particular be an oil composition produced by microorganisms. Preferably, methylene substituted lipids directly issued from extraction of aforesaid microorganisms are obtained in more than 5 wt %, preferably more than 10 wt %, more preferably more than 20 wt %, even more preferably more than 30 wt %, with other lipids either in the form of triglycerides or in the form of phospholipids. Triglycerides containing methylene lipids may be first hydrolysed to obtain free fatty acids and side products such as glycerol. Resulting free fatty acids are separated according to well-known methods in the art such as liquid-liquid extraction with solvent and water.

In one embodiment, step b) includes reacting the 10-methylene-substituted lipid(s), optionally in mixture with other lipids, with a reactant containing bromine under conditions to provide the corresponding 10-bromomethyl lipid(s), optionally in mixture with said other lipids. These lipids may also have reacted to form brominated lipids. Such 10-bromomethyl lipid is particularly useful for preparing further products such as the corresponding 10-aminomethyl lipid or 10-hydroxymethyl lipid. In this respect, reacting the 10-methylene-substituted lipid(s) with a reactant containing bromine is preferably made according to the procedure described in the examples and comparative examples of EP3030543, to Arkema.

Advantageously, step b) may further include reacting the 10-bromomethyl lipid(s), optionally in mixture with other lipids, with a reactant containing an amine under conditions to provide the corresponding 10-aminomethyl lipid(s), optionally in mixture with said other lipids. These lipids may also have reacted to form lipids with amino group. The 10-aminomethyl lipid(s) thus obtained is particularly useful for preparing further products such as lactams.

Alternatively, step b) may further include reacting the 10-bromomethyl lipid(s), optionally in mixture with other lipids, with a reactant containing a base under conditions to provide the corresponding 10-hydroxymethyl lipid(s), optionally in mixture with said other lipids. These lipids may also have reacted to form lipids with hydroxyl group. The 10-hydroxymethyl lipid thus obtained is particularly useful for preparing further products such as lactones.

Such lactams and lactones may be used as such or may be used as starting material for the same purpose. Lactams and lactones may also be used for making, respectively polyamides and polyesters via ring opening polymerisation (ROP).

Advantageously, the process of the invention further may comprise a purification step c) wherein the 10-bromomethyl lipid(s), 10-aminomethyl lipid(s), 10-hydroxymethyl lipid(s), lactone(s) or lactam(s) obtained from step b) is submitted to a purification step to separate the reaction products if the starting material is a mixture and/or to separate isomers or enantiomers formed.

Starting material and each of the products described herein bearing functional groups chosen among bromine, amino, hydroxyl, lactone and lactame may be purified using liquid chromatography techniques using appropriate solvents and solid phases (e.g. silica gel, alumina) as well as technology (e.g. column, thin layer, or centrifugal chromatography). If enantiomer separation is desired, differential crystallization may be applied with an appropriate chiral aid, preferably to make a salt, preferably using a chiral amine or alcohol, or a chiral acid in case the 10-methylene substituted lipid contains an amine moiety. Alternative purification method includes the use of liquid chromatography with the aid of a chiral stationary phase according to conventional methods.

In case the product to be purified is a carboxylic ester, a lactone or a lactam, distillation is preferred, unless enantiomers separation is desired.

Advantageously, when the 10-methylene-substituted lipid(s) provided in step a) has a carboxylic acid as terminal group, the process may further comprise an esterification step comprising contacting the methyl-substituted lipid(s) obtained from step b), optionally in mixture with other lipids and/or purified, with an excess of alcohol under conditions to provide the corresponding ester.

Advantageously, when the 10-methylene-substituted lipid(s) provided in step a) has a carboxylic acid ester as terminal group, the process may further comprise an hydrolysis step comprising contacting the 10-methyl-substituted lipid(s) obtained from step b), optionally in mixture with other lipids and/or purified, with an excess of water under conditions to provide the corresponding carboxylic acid.

According to a further aspect, the invention provides a process for preparing polyamide materials comprising submitting at least one intermediate product of the present invention or a composition of the present invention, said at least one intermediate product having an amino group as functional group and a carboxylic acid as terminal group, to polycondensation conditions, alone or in presence of another monomer containing at least one amino group and one carboxylic acid group, and obtaining polyamide homo-oligomers, homo-polymers, co-oligomers or co-polymers.

According to a further aspect, the invention provides a process for preparing copolymers comprising the following steps:
submitting at least one intermediate product of the present invention or a composition of the present invention, said at least one intermediate product having an amino group as functional group and a carboxylic acid as terminal group, to polycondensation conditions, alone or in presence of another monomer containing at least one amino group and one carboxylic acid group, and obtaining polyamide homo-oligomers, homo-polymers, co-oligomers or co-polymers,
and contacting the polyamide homo-oligomers, homo-polymers, co-oligomers or co-polymers with a polyether under conditions to obtain the grafting of said polyether on the free amine or free carboxylic acid moiety of the said polyamide.

According to a further aspect, the invention provides a process for preparing lactone comprising submitting at least one intermediate product of the present invention or the composition of the present invention, said at least one intermediate product having an hydroxyl group as functional group and a carboxylic acid as terminal group, to ring formation conditions to obtain a lactone.

According to a further aspect, the invention provides a lactone, more specifically 11-alkyloxacyclododecan-2-one, in particular obtained from the previously mentioned process, wherein the alkyl group branched in the Δ11 position of the ring is a linear aliphatic chain having 4 to 10 carbon atoms, preferably 6 to 8 carbon atoms, most preferably 8. Preferably, the aliphatic chain is saturated.

According to a further aspect, the invention provides a process for preparing lactam comprising submitting at least one intermediate product of the present invention or the composition of the present invention, said at least one intermediate product having an amino group as functional group and a carboxylic acid as terminal group, to ring formation conditions to obtain a lactam.

According to a further aspect, the invention provides a lactam, more specifically 11-alkylazacyclododecan-2-one, in particular obtained from the previously mentioned process, wherein the alkyl group branched in the Δ11 position of the ring is a linear aliphatic chain having 4 to 10 carbon atoms, preferably 6 to 8 carbon atoms, most preferably 8. Preferably, the aliphatic chain is saturated.

The above mentioned lactams and lactones can be advantageously used as flavors, fragrances, or precursors for preparing lactams and lactones.

According to a further aspect, the invention provides a process for preparing anti-fungal compounds comprising contacting at least one starting material, one intermediate product of the present invention or the composition of the present invention, said at least one starting material or one intermediate product having a carboxylic acid as terminal group, with a metal or metal salt under conditions to obtain carboxylic metal salt of said one starting material or said one intermediate product. Such metal is preferably zinc.

### DETAILED DESCRIPTION OF THE INVENTION

For the purpose of the invention, the following definitions are given:

As used herein, the term “corresponding” in “corresponding 10-bromomethyl lipid” means that the 10-bromomethyl lipid has an aliphatic linear chain with a methyl group being in the Δ10 position and with the same number of carbon atoms than the aliphatic linear chain of the 10-methylene-substituted lipid from which the 10-bromomethyl lipid is obtained. Similarly, a “corresponding 10-aminomethyl lipid” has the same aliphatic linear chain with a methyl group being in the Δ10 position than the 10-bromomethyl lipid from which it is obtained, the bromine being replaced by the amino group, and a “corresponding 10-hydroxymethyl lipid” has the same aliphatic linear chain with a methyl group being in the Δ10 position than the 10-bromomethyl lipid from which it is obtained, the bromine being replaced by the hydroxyl group.

As used herein, in the expression “C#”, “#” is a positive integer which corresponds to the number of carbon atoms.

When it is mentioned that a linear aliphatic chain has X carbon atoms, this number X of carbon atoms excludes any carbon branched on the linear aliphatic chain. For example, an aliphatic linear chain of 14 carbon atoms with a branched methyl group has a total of 15 carbon atoms.

The term “ester” here refers to carboxylic acid esters.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all numbers and fractions subsumed within the respective ranges, as well as the recited endpoints.

All documents cited in the present specification are hereby incorporated by reference in their entirety. In particular, the teachings of all documents herein specifically referred to are incorporated by reference.

With “bio-organic compound” or “microbial-derived organic The particular features, structures, characteristics or embodiments may be combined in any suitable manner, as would be apparent to a person skilled in the art from this disclosure, in one or more embodiments.

The invention provides a process for preparing intermediate products and also further products from these intermediate products.

### Method for Preparing the Intermediate Product of the Invention

### Step a)

Each of the 10-methylene-substituted lipid(s) provided in step a) comprises an aliphatic linear chain of 14-20 carbon atoms having a methylene (═CH₂) group branched in the Δ10 position and a terminal group chosen from a carboxylic acid and a carboxylic acid ester.

Advantageously, the aliphatic linear chain has 16-18 carbon atoms. In a preferred embodiment, the aliphatic linear chain has 18 carbon atoms. Such aliphatic linear chain is preferably saturated.

The 10-methylene-substituted lipid(s) may be chosen from 10-methylenestearic acid or ester, 10-methylenepalmitic acid or ester and 10-methylenearachidic acid or ester, preferably 10-methylenestearic acid or ester.

Preferred esters are methyl, ethyl, propyl and isopropyl esters.

In one embodiment, the 10-methylene-substituted lipid(s) is(are) produced by microorganisms. Step a) then includes cultivating an appropriate cell culture and recovering an oil composition from the cell culture, said oil composition comprising 10-methylene-substituted lipid(s) as previously defined.

An appropriate cell is a cell comprising a methyltransferase gene and/or a reductase gene for producing branched methyl lipids or compositions that include such lipids as disclosed in WO2018/057607, incorporated therein by reference.

A methyltransferase gene encodes a methyltransferase protein, which is an enzyme capable of transferring a carbon atom and one or more protons bound thereto from a substrate such as S-adenosyl methionine to a fatty acid such as oleic acid (e.g., wherein the fatty acid is present as a free fatty acid, carboxylate, phospholipid, diacylglycerol, or triacylglycerol). A reductase gene encodes a reductase protein, which is an enzyme capable of reducing, often in an NADPH-dependent manner, a double bond of a fatty acid (e.g., wherein the fatty acid is present as a free fatty acid, carboxylate, phospholipid, diacylglycerol, or triacylglycerol).

Such cell usually comprises nucleic acids comprising the above mentioned methyltransferase gene and/or a reductase gene and is generally a cell transformed by any suitable well known technique including, e.g., biolistics, electroporation, glass bead transformation, and silicon carbide whisker transformation.

Lists of appropriate cells and nucleic acids are given in WO2018/057607. The cell may be selected from the group of algae, bacteria, molds, fungi, plants and yeasts. By way of example, suitable cells include, but are not limited to, Saccharomyces cerevisiae, Yarrowia lipolytica, or Arxula adeninivorans.

Typically, manufacturing a lipid in a microorganism involves growing microorganisms which are capable of producing a desired lipid in a fermentor or bioreactor and recovering the oil produced by the cells either accumulated within the cells or secreted by the cells. The culture medium and conditions can be chosen based on the species of the cell to be cultured and can be optimized to provide for maximal production of the desired lipid profile. Any known method suitable for recovering an oil composition from a culture of cells can be used, such as harvesting cultured cells, such as by filtration or centrifugation, lysing cells to create a lysate containing the lipids accumulated within the cells or collecting the secreted lipids from the culture medium, and extracting the lipid/hydrocarbon components using a hydrophobic solvent.

Step a) may comprise cultivating appropriate cells in a bioreactor, in a culture medium optionally supplemented with an unbranched, unsaturated fatty acid, such as oleic acid, that serves as a substrate for methylation. The culture medium may optionally be supplemented with methionine or s-adenosyl methionine, which may similarly serve as a substrate. Thus, step a) may comprise contacting a cell or plurality of cells with oleic acid, methionine, or both. Step a) may comprise recovering lipids from the cells and/or from the culture medium, such as by extraction with an organic solvent.

The recovered lipids form an oil composition which may contain at least 75%, 80%, 85%, 90%, 95%, or 99% by weight of lipids, mainly as triglycerides, and to a lesser extent, as diglycerides, monoglycerides, phospholipids and free carboxylic acids. The oil composition comprises branched methylene-substituted lipids, and optionally (methyl)lipids. The methylene-substituted lipids may contain a free carboxylic acid (e.g., 10-methylenestearic acid, 10-methylenepalmitic acid, 12-methyleneoleic acid, 13-methyleneoleic acid, 10-methylene-octadec-12-enoic acid).

The recovered oil composition can be directly sent to the step b) or step a) may comprise submitting the oil composition to conditions suitable for recovering one or more 10-methylene-substituted lipids with an aliphatic linear chain of 14-20 carbon atoms having a methylene (═CH₂) group branched in the Δ10 position and a terminal group chosen from a carboxylic acid and a carboxylic acid ester or a mixture of such methylene-substituted lipids. By way of example, vacuum distillation may be used for recovering the one or more 10-methylene-substituted lipids in the form of carboxylic acids. In this case, the carboxylic acids are obtained from the triglyceride containing crude oil composition by hydrolysis in the presence of an acidic catalyst such as sulfuric, hydrochloric or phosphoric acid.

### Step b)

The 10-methylene-substituted lipid, mixture of 10-methylene-substituted lipids or oil composition containing 10-methylene-substituted lipid(s) provided in step a) is then submitted to step b) in which it is reacted with one or several reagents under conditions to give the corresponding 10-methyl-substituted lipid(s) comprising a functional group bound to the carbon of the branched methyl group. Such functional group is bromo (—Br).

This bromo functional group may be further substituted with a hydroxyl functional group to afford a 10-hydroxymethyl lipid. Alternatively, the bromo functional group may be substituted with an amino functional group to afford a 10-aminomethyl lipid. The said amino functional group may be a monosubstituted amino group such as methylamino or ethylamino or a disubstituted amino group such as diethylamino, diisopropylamino, pyrrolidinyl or methylethylamino.

A preferred functional group is chosen from bromo (—Br), hydroxyl (13 OH), amino (—NH₂) groups. Other amino groups are mono or disubstituted amino groups.

In particular, the functional group can be chosen depending on the reaction in which the intermediate compound is intended to be used.

Such “corresponding 10-methyl-substituted lipid” comprises an aliphatic linear chain with the same number of carbon atoms than the aliphatic linear chain of the starting methylene-substituted lipid and a methyl group branched in the Δ10 position.

Appropriate conditions for obtaining the above methyl-substituted lipids can be determined by those skilled in the art of chemical synthesis from general knowledge. In particular, one of the chosen reactants contains the functional group which will react with the carbon-carbon double bond in the 10 position of the 10-methylene-substituted lipid to give the 10-methyl-substituted lipid(s) with the functional group.

As already mentioned, if other lipids are present which also contain one or several carbon-carbon double bonds, such double bonds may also react with the functional group and give a lipid having the same functional group as the 10-methyl-substituted lipid. On the contrary, lipids without carbon-carbon double bond will probably not react.

We may then obtain a composition containing 10-methyl-substituted lipid(s) having the functional group in the Δ10 position, lipids with one or several of the same functional group and other lipids without this functional group. Depending on the intended use of such composition, separation of some of the lipids not of interest may be envisaged, for example by distillation (atmospheric and/or vacuum distillation) or any other appropriate separation techniques.

### Composition

The composition of the present invention contains at least one intermediate product as previously defined, in particular obtained by the process described therein.

The composition preferably contains at least 25 wt %, 30 wt %, 35%, or more of one or more intermediate products, up to 90% wt, 95% wt, 99% wt. The intermediate product(s) content may be within a range defined by any combination of the above limits.

Such composition may in particular contain in addition other lipids in content from 1% wt, 5% wt, 10% wt, and up to 75% wt, 70% wt, 65% wt, or within a range defined by any combination of these limits.

The lipids may comprise C12-C30 fatty acids. Generally, lipids comprise C14-C24 fatty acids. Such lipids can be present as fatty acids, triacylglycerides (TAG) or ester of fatty acids.

The nature and proportions of lipids can be determined by chromatography techniques, in particular gas chromatography coupled with mass spectrometry or by NMR. Typically, crude oil composition issued from genetically modified yeast fermentation contains 10-40 wt % of 10-methylenestearic acid, 1-10 wt % of 10-methylenepalmitic acid along with 0.1-1 wt % of 10-margaric acid, in the form of triglycerides.

### EXAMPLES

The embodiments of the present invention will be better understood by looking at the different examples below, illustrated by the following figures showing preferred pathways and products preparation schemes and methods. On all the figures, R represents an alkyl group, for example chosen from a methyl, ethyl, propyl and isopropyl group.

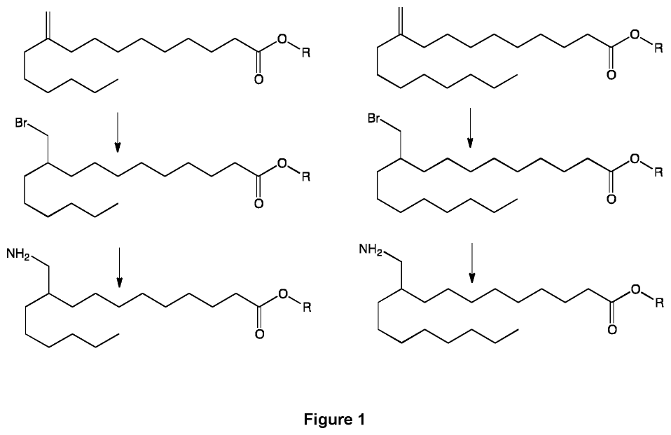

FIG. 1 represents a pathway from 10-methylene-palmitic ester/acid to 10-bromomethyl-palmitic ester/acid and 10-aminomethyl-palmitic ester/acid (left side), and 10-methylene-stearic ester/acid to 10-bromomethyl-stearic ester/acid and 10-aminomethyl-stearic ester/acid (right side). R may represent an alkyl or hydrogen group.

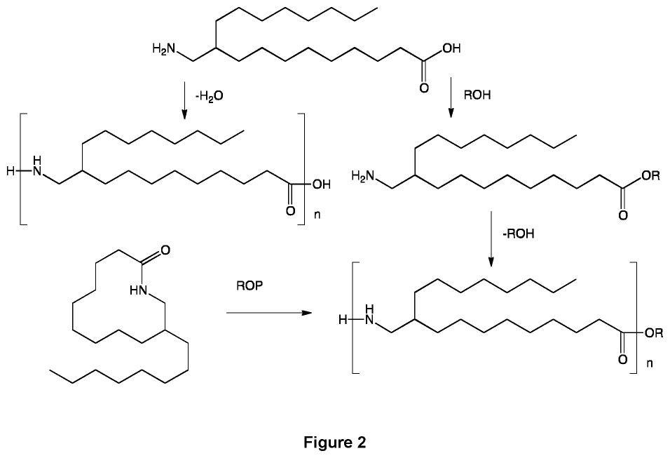

FIG. 2 represents the polycondensation of 10-aminomethyl-stearic acid to a polyamide, directly with water elimination or via the formation of the corresponding 10-aminomethyl-stearic ester. In the figure, n represents the number of units of the polyamide. Also shown on bottom left is ring opening polymerization (ROP) of 11-octylazacyclododecan-2-one.

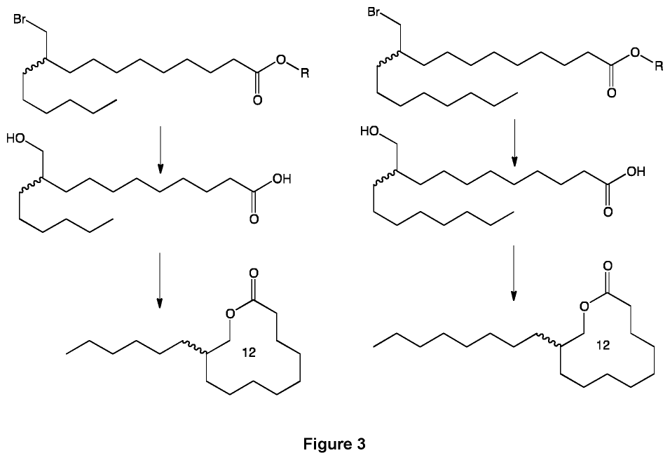

FIG. 3 represents the cyclisation of 10-hydroxymethylstearic acid to the corresponding lactone, 11-octyloxacyclododecan-2-one (right side) and cyclization of 10-hydroxymethylpalmitic acid to the corresponding lactone, 11-hexyloxacyclododecan-2-one (left side).

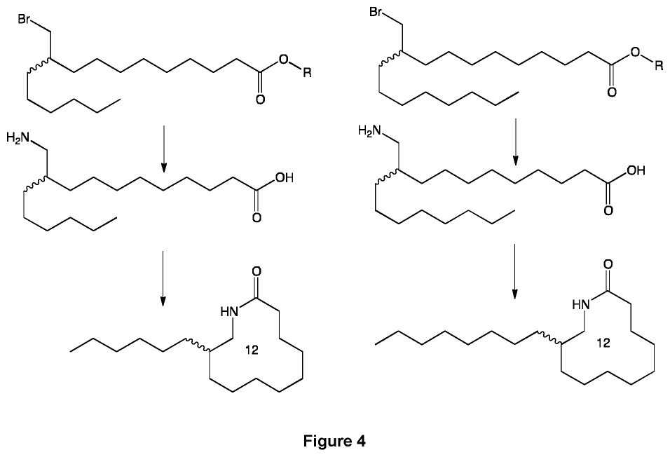

FIG. 4 represents the cyclisation of 10-aminomethylstearic acid to the corresponding lactam, 11-octylazacyclododecan-2-one (right side) and cyclization of 10-aminomethylpalmitic acid to the corresponding lactam, 11-hexylazacyclododecan-2-one (left side).

### Bromination of 10-Methylene Stearic Acid or Esters

Hydrobromination of 10-methylene-stearic acid can be done in the presence of hydrobromic acid and a generator of radical species, which can be ultra-violet light (UV), chemical species such as oxygen, ozone, diazo compounds, peroxides such as hydrogen peroxide, di-benzoyl-peroxide, di-ter-butyl-peroxide, 4-chlorophenyl-perbenzoic acid or perbenzoic acid.

Preferred hydrobromination methods include those described in WO2015/019028 (EP3030543B1), FR951932 and CN1100030C, which make use of hydrobromic acid and oxygen, and wherein the olefinic product to be hydrobrominated is in solution within a suitable solvent.

A suitable solvent means a solvent which does not act as scavenger of radicals that are formed during the reaction.

Suitable solvents include those disclosed in WO2015/019028 such as, but not limited to, benzene, fluorobenzene, chlorobenzene, toluene, methylcyclohexane, trifluorotoluene (“BTF”), ethylbenzene, xylene, cyclohexane, methylcyclohexane, heptane, isooctane, 1,1,1-trichloroethane and dibromomethane, and mixtures thereof. Aromatic solvents such as benzene, toluene or xylene, and also halogenated solvents such as monochlorobenzene, are generally described as “good solvents”. The term “good solvent” is intended to mean a solvent which allows miscibility and solubility of the reagents and of the products of the hydrobromination reaction at the hydrobromination reaction temperature, and which thus provides the system with better reactivity. Miscibility usually denotes the capacity of various liquids to mix together. If the mixture obtained is homogeneous, the liquids are described as miscible for the purposes of the invention. The solubility of an ionic or molecular compound, called solute, is the maximum concentration (in moles per liter) of this compound that can be dissolved or dissociated in a solvent, at a given temperature.

By allowing total solubility of the reagents and products, a good solvent also prevents blockages in the equipment. Conversely, solvents such as cyclohexane, methylcyclohexane or heptane are generally categorized as “poor solvents” in this type of hydrobromination process.

Reaction temperature is from −50° C. to +20° C. so as to keep regioselectivity of bromine atom radical addition onto terminal (unsubstituted) sp2 carbon atom of the olefin functional group. Advantageously, in order to have HBr and the reagent soluble in the solvent, the temperature at which the solvent is used is preferably between the highest temperature of crystallization of the reagents and the limiting temperature of the anti-Markovnikov reaction, i.e. in the range of from −50° C. to 20° C.

Esters of 10-methylenestearic acid can undergo the same reaction without significant modification of preparation method.

Resulting 10-bromomethylstearic acid/ester is then reacted with ammonia under mild conditions to provide 10-bromomethylstearic acid/ester as shown in FIG. 1.

### Preparation of Amino Derivatives

10-aminomethylstearic acid/ester can be prepared according to methods described in WO2010/070228 (EP2358662B1), FR988699 and GB591027 which are dedicated to 11-aminoundecanoic acid preparation from contacting 11-bromoundecanoic acid with an ammonia solution. The method from WO2010/070228 is preferred.

### Hydroxylation of Bromo-Substituted Methylstearic, Margaric and Palmitic Acid/Esters

10-bromomethylstearic, margaric or palmitic acid/ester or their mixture can undergo bromine exchange under acidic or basic conditions in the presence of water to afford, respectively, 10-hydroxymethylstearic, margaric and palmitic acid/ester or their mixture in high yield. Care should be taken 1) to avoid using an acid or a base which would be susceptible to nucleophilic exchange with bromine atom, such as amines and strong carboxylic or sulphonic acids such as trifluoromethylsulphonic acid, and 2) to control reaction temperature to avoid undesired elimination reaction, which would lead back to starting material, i.e. to 10-methylenestearic, margaric or palmitic acid or to isomers such as 10-methyloleic acid.

### Separation of Enantiomers

If desired, 10-hydroxymethylstearic acid/ester, 10-bromomethylstearic acid/ester and 10-aminomethylstearic acid/ester can be each independently purified to separate their enantiomers. Methods of separation include, without limitation, differential crystallization with a chiral molecule, separation by liquid phase chromatography on a chiral support or with a chiral solvent. With regards to 10-aminomethylstearic acid and 10-aminomethylstearic acid esters, preferred method of separation include formation of a salt with a chiral acid such as (+/−)-tartaric acid.

The same method can be used to separate enantiomers of 10-hydroxymethylpalmitic acid/ester, 10-bromomethylpalmitic acid/ester, 10-aminomethypalmitic acid/ester, 10-hydroxymethyl margaric acid/ester, 10-bromomethylmargaric acid/ester and 10-aminomethymargaric acid/ester.

### Esters of Stearic, Margaric and Palmitic Acid Derivatives

Esters of here described stearic, margaric and palmitic acid derivatives may be prepared using conventional methods by contacting a stearic, margaric or palmitic acid derivative or their mixture with an excess of an alcohol to be esterified, in the presence of a strong acid such as sulfuric acid. Preferably, produced water is removed from the reaction media to limit reverse reaction of hydrolysis. Preferred alcohols include methanol, ethanol, propanol and isopropanol. Preferred esters include methyl, ethyl, propyl and isopropyl esters.

Esters of here described stearic, margaric and palmitic acid derivatives may be hydrolyzed to the corresponding stearic, margaric and palmitic acid derivative by contacting it with an excess of water in the presence of a strong acid. Produced alcohol is preferably removed from the reaction media to limit reverse reaction of esterification.

### Polyamides Preparation

10-aminomethyl-stearic acid can be condensed to provide polyamide or oligomers thereof, as shown in FIG. 2.

Reaction can be conducted without solvent or catalyst at 120-180° C. for a period of time adapted to the desired properties of the polymer. Evolved water is removed from the reaction media as it forms by distillation.

10-aminomethylstearic acid esters can also be condensed using the same method. Evolved alcohol is removed accordingly.

Suitable method of polymerization can be found in WO2000/68298, where hyperbranched polyamides are prepared. This document shows that hyperbranched polyamides improve transparency, which is a key factor to displace the PMMA (PolyMethylMethAcrylate) market for e.g. automotive lighting.

The same method may be applied to polycondensation of 10-aminomethyl-margaric or 10-aminomethyl-palmitic acid monomers.

### Copolymers Preparation

Preparation of copolymers of 10-aminomethyl-stearic acid with other monomers such as 11-aminoundecanoic acid, 12-aminododecanoic acid, 10-aminodecanoic acid may be envisioned using the same methods as above.

Other important market uses for high performance copolymers include polyether block amides (PEBA), which are thermoplastic elastomers obtained by 1) polycondensation of homopolymer of an aminoacid according to the invention or of a copolymer of the same with one of the monomers used for making PA6, PA11 and PA12 followed by terminal grafting of a polyether on the free amine or free carboxylic acid moiety of the polyamide. Known PEBA tradenames include PEBAX® (Arkema) and VESTAMID® (Evonik Industries).

It is anticipated that introducing the monomers according to the invention as co-monomers in the preparation of PEBAX or Vestamide derivatives could result in better:
1) resistance to UV degradation since the monomer possesses tertiary alkyl moiety, which is known to stabilize radicals. Therefore, claiming UV resistance improvement could be an option.
2) Improved thermal resistance at low temperatures since polyamide crystallinity would be lowered by introduction of a monomer according to the invention,
3) Improved transparence

By way of example, 10-aminomethylstearic acid can be poly-condensed to afford polyamide similar to polyamide 12 (Nylon 12®) with a straight C6 side chain. Copolymerization with 12-aminododecanoic acid allows mitigation of Nylon 12® physical properties. Other closely related polyamides include Nylon 11, marketed by Arkema under the trade name Rilsan®.

10-hydroxymethylstearic acid can be poly-condensed to afford polyester.

The same method may be applied to copolymerization of 10-aminomethyl-margaric or 10-aminomethyl-palmitic acid monomers.

### Lactone and Lactam Preparation

Lactone ring formation from 10-bromomethylstearic acid, 10-bromomethylmargaric acid and 10-bromomethylpalmitic acid can be performed in the presence of K₂CO₃ by heating at ca. 100° C. in DMSO (dimethylsulfoxide), according to the method described by Galli C. and Mandolini in Organic Syntheses, (1978), 58, 98-101, “Macrolides from cyclization of ω-bromocarboxylic acids: 11-hydroxyundecanoic lactone”.

Lactone and lactam ring formation from, respectively, 10-hydroxymethylstearic acid, 10-hydroxymethylmargaric acid, 10-hydroxymethylpalmitic acid and 10-aminomethylstearic acid, 10-aminomethylmargaric acid, 10-aminomethylpalmitic acid, can be done by e.g. heating under high dilution in an appropriate solvent. To this aim, lactone ring formation from hydroxyacids can be done according to the method from Chemishe Berichte, (1947), 80, 129-137, wherein hydroxyacid is cyclized in boiling methylethylketone (MEK) in the presence of K₂CO₃ to afford desired lactone, or according to Mukaiyama et al. in Chemistry Letters, (1976), (1), 49-50, wherein hydroxyacid is cyclized by heating in trimethylamine in the presence of 2-chloro-1-methylpyridinium iodide. Lactam ring formation from aminoacids can be performed according to Yunxin Bo and Donglu Bai, in Chinese Chemical Letters, (1994), Vol. 5, issue 2, 95-96, wherein aminoacid is cyclized in the presence of 2-bromo-1-methylpyridinium iodide.

Alternatively, lactone and lactam ring formation may be performed by 1) anchoring of the starting material on a solid phase polymeric support bearing an appropriate linking group, then 2) cleavage of the starting material which, at cleavage step, cyclizes into the corresponding lactone or lactam.

Resulting 11-hexyloxacyclododecan-2-one or 11-octyloxacyclododecan-2-one (FIG. 3) and 11-hexylazacyclododecan-2-one or 11-octylazacyclododecan-2-one (FIG. 4) may find use as such or as starting material for making flavors and fragrances, as some lighter lactones and cyclic ethers are known to have e.g. peach, mango or musk odor, as starting material for making compositions suitable for crop protection, in particular as mimic or antagonist of sexual hormone receptors of bugs or other insects, or as monomers or co-monomers for preparing polyesters or polyamides via Ring Opening Polymerization (ROP) using conventional techniques.

### Preparation of Metal Salts with Anti-Fungal Properties

10-hydroxymethylstearic, 10-hydroxymethylmargaric and 10-hydroxymethylpalmitic acid can be reacted with an appropriate metal or metal salt to obtain corresponding 10-hydroxymethylstearic, 10-hydroxymethylmargaric and 10-hydroxymethylpalmitic acid metal salt as anti-fungal compounds useful in human and animal therapy as well as for crop protection. Preferred metal is zinc. Closest therapeutic product is Mycodecyl® (zinc undecylenate).

1. Intermediate product for preparing a product chosen at least from polyamides, polyesters, lactams and lactones, and optionally said intermediate product having a saturated aliphatic linear chain of 14-20 carbon atoms, said aliphatic linear chain having
a terminal group chosen from a carboxylic acid and a carboxylic acid ester,
a methyl group branched in the Δ10 position,
a functional group bound to the carbon of the said branched methyl group, this functional group being chosen from bromo (—Br), hydroxyl (—OH) and amino (—NH₂) groups, wherein bromo (—Br) is preferred.

2. Intermediate product according to claim 1, wherein the aliphatic linear chain has 16, 17 or 18 carbon atoms, preferably, 16 or 18.

3. Intermediate product according to any one of claim 1 or 2, wherein the terminal group is an ester chosen from methyl, ethyl, propyl and isopropyl esters.

4. Composition comprising at least one intermediate product of any one of claims 1 to 3, optionally in mixture with other lipids.

5. Process for preparing the intermediate product of any one of claims 1 to 3 or the composition of claim 4, said process comprising the following steps:
a) providing at least one 10-methylene-substituted lipid comprising an aliphatic linear chain of 14-20 carbon atoms having a methylene (═CH₂) group branched in the Δ10 position and a terminal group chosen from a carboxylic acid and a carboxylic acid ester, optionally in mixture with other lipids,
b) reacting said at least one 10-methylene-substituted lipid, optionally in mixture with other lipids, with at least one reagent under conditions to provide at least one corresponding 10-methyl-substituted lipid, optionally in mixture with said other lipids, said corresponding 10-methyl-substituted lipid comprising :
a saturated aliphatic linear chain with the same number of carbon atoms than the aliphatic linear chain of the 10-methylene-substituted lipid,
a methyl group branched in the Δ10 position, and
a functional group bound to the carbon of the branched methyl group, chosen from bromo (—Br), hydroxyl (—OH) and amino (—NH₂) groups, preferably bromo (—Br).

6. Process according to claim 5, wherein step b) includes reacting the 10-methylene-substituted lipid(s), optionally in mixture with other lipids, with a reactant containing bromine under conditions to provide the corresponding 10-bromomethyl lipid(s), optionally in mixture with said other lipids.

7. Process according to claim 5 or 6, wherein step b) further includes reacting the 10-bromomethyl lipid(s), optionally in mixture with other lipids, with a reactant containing an amine under conditions to provide the corresponding 10-aminomethyl lipid(s), optionally in mixture with said other lipids.

8. Process according to claim 5 or 6, wherein step b) further includes reacting the 10-bromomethyl lipid(s), optionally in mixture with other lipids, with a reactant containing a base under conditions to provide the corresponding 10-hydroxymethyl lipid(s), optionally in mixture with said other lipids.

9. Process according to any one of claims 5 to 8, further comprising a purification step c) wherein the 10-bromomethyl lipid(s), 10-aminomethyl lipid(s) or 10-hydroxymethyl(s) lipid obtained from step b) is submitted to a purification step to separate the enantiomers formed.

10. Process according to any one of claims 5 to 9, wherein
the 10-methylene-substituted lipid(s) provided in step a) has a carboxylic acid as terminal group, and the process further comprises an esterification step comprising contacting the 10-methyl-substituted lipid obtained from step b), optionally in mixture with other lipids and/or purified, with an excess of alcohol under conditions to provide the corresponding ester, or
the 10-methylene-substituted lipid(s) provided in step a) has a carboxylic acid ester as terminal group, and the process further comprises an hydrolysis step comprising contacting the 10-methyl-substituted lipid(s) obtained from step b), optionally in mixture with other lipids and/or purified, with an excess of water under conditions to provide the corresponding carboxylic acid.

11. Process for preparing polyamide materials comprising submitting at least one intermediate product of any one of claims 1 to 3 or the composition of claim 4, said at least one intermediate product having an amino group as functional group and a carboxylic acid as terminal group to polycondensation conditions, alone or in presence of another monomer containing at least one amino group and one carboxylic acid group, and obtaining polyamide homo-oligomers, homo-polymers, co-oligomers or co-polymers.

12. Process for preparing copolymers comprising the following steps:
submitting at least one intermediate product of any one of claims 1 to 3 or the composition of claim 4, said at least one intermediate product having an amino group as functional group and a carboxylic acid as terminal group to polycondensation conditions, alone or in presence of another monomer containing at least one amino group and one carboxylic acid group, and obtaining polyamide homo-oligomers, homo-polymers, co-oligomers or co-polymers,
and contacting the polyamide homo-oligomers, homo-polymers, co-oligomers or co-polymers with a polyether under conditions to obtain the grafting of said polyether on the free amine or free carboxylic acid moiety of the said polyamide.

13. 11-alkyloxacyclododecan-2-one, in particular obtained from submitting at least one intermediate product of any one of claims 1 to 3 or the composition of claim 4, said at least one intermediate product having an hydroxyl group as functional group and a carboxylic acid as terminal group, to ring formation conditions to obtain a lactone, wherein the alkyl group branched in the Δ11 position of the ring is a linear aliphatic chain having 4 to 10 carbon atoms, preferably 6, 7 or 8 carbon atoms, most preferably 8.

14. 11-alkylazacyclododecan-2-one, in particular obtained from submitting at least one intermediate product of any one of claims 1 to 3 or the composition of claim 4, said at least one intermediate product having an amino group as functional group and a carboxylic acid as terminal group, to ring formation conditions to obtain a lactam, wherein the alkyl group branched in the Δ11 position of the ring is a linear aliphatic chain having 4 to 10 carbon atoms, preferably 6, 7 or 8 carbon atoms, most preferably 8.
